SEQUENCE LISTING (4 sequences)

SEQ ID NO: 1 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - DNA, 20 bp, Synthetic construct
  source 1..20: mol_type = other DNA
gaccccaaaatcagcgaaat

SEQ ID NO: 3 - DNA, 24 bp, Synthetic construct
  source 1..24: mol_type = other DNA
tctggttactgccagttgaatctg

SEQ ID NO: 4 - DNA, 24 bp, Synthetic construct
  source 1..24: mol_type = other DNA
accccgcattacgtttggtggacc

(19) United States
(12) Patent Application Publication
(10) Pub. No.: US 2024/0218056 A1
(43) Pub. Date: Jul. 4, 2024

(54) NUCLEOCAPSID-SPECIFIC ANTIBODIES AND METHODS FOR THE TREATMENT AND PREVENTION OF SARS-COV-2 INFECTION THEREWITH

(71) Applicant: Northwestern University, Evanston, IL (US)

(72) Inventor: Pablo Penaloza-MacMaster, VIII, Evanston, IL (US)

(21) Appl. No.: 18/530,063

(22) Filed: Dec. 5, 2023

Related U.S. Application Data
(60) Provisional application No. 63/386,080, filed on Dec. 5, 2022.

(51) Int. Cl. C07K 16/10; A61K 39/00; A61P 31/14

(52) U.S. Cl. CPC C07K 16/1003; A61P 31/14; A61K 2039/505; C07K 2317/55; C07K 2317/56; C07K 2317/622

(57) ABSTRACT

Provided herein are compositions and methods for the treatment and/or prevention of SARS-COV-2 infection, and symptoms and conditions associated therewith (e.g., COVID-19). In particular, provided herein are nucleocapsid-specific antibodies and methods of use thereof therapeutically or prophylactically to enhance the clearance of SARS-COV-2 infection from a subject.

### CROSS-REFERENCE TO RELATED APPLICATIONS

The present invention claims priority to U.S. Provisional Patent Application Ser. No. 63/386,080, filed Dec. 5, 2022, which is incorporated by reference in its entirety.

### SEQUENCE LISTING

The text of the computer readable sequence listing filed herewith, titled “NWEST-41491.202_SequenceListing.xml”, created Mar. 20, 2024, having a file size of 5,030 bytes, is hereby incorporated by reference in its entirety.

### FIELD

Provided herein are compositions and methods for the treatment and/or prevention of SARS-COV-2 infection, and symptoms and conditions associated therewith (e.g., COVID-19). In particular, provided herein are nucleocapsid-specific antibodies and methods of use thereof therapeutically or prophylactically to enhance the clearance of SARS-COV-2 infection from a subject.

### BACKGROUND

Coronavirus disease 2019 (COVID-19) is caused by infection of a subject (e.g., human or other susceptible organism) by severe acute respiratory syndrome coronavirus 2 (SARS-COV-2). SARS-COV-2 has infected more than 600 million people and continues to spread around the globe. Although vaccines and monoclonal antibody therapies can help prevent severe disease and death, breakthrough infections can occur, highlighting the need for improving current vaccines and available treatments (Refs. 1-9; incorporated by reference in their entireties). The SARS-COV-2 spike protein is critical for viral entry, making this protein an important antigen present in all SARS-COV-2 vaccines and the only target for all monoclonal antibody therapies. Besides spike-specific immune responses, other antigen-specific immune responses are elicited during natural SARS-COV-2 infection (Refs. 10-13; incorporated by reference in their entireties), but their roles in protecting against infection has remained unclear. In particular, it is unknown in the field whether antibodies specific to internal viral proteins such as the nucleocapsid protein, which does not play a role in viral entry, can confer protection against SARS-COV-2. Knowing if other antigen-specific antibodies are protective could facilitate the development of more potent vaccines and monoclonal antibody therapies for coronavirus infections.

### SUMMARY

Provided herein are compositions and methods for the treatment and/or prevention of SARS-COV-2 infection, and symptoms and conditions associated therewith (e.g., COVID-19). In particular, provided herein are nucleocapsid-specific antibodies and methods of use thereof therapeutically or prophylactically to enhance the clearance of SARS-COV-2 infection from a subject.

In some embodiments, provided herein are pharmaceutical compositions comprising (i) an antibody or antigen-binding fragment capable of binding to one or more epitopes on a SARS-CoV-2 nucleocapsid protein and preventing, reducing the likelihood, and/or reducing the severity or length of a SARS-COV-2 infection when administered to a subject, and (ii) a pharmaceutically acceptable carrier. In some embodiments, the pharmaceutical composition is formulated for oral or parenteral administration. In some embodiments, the antibody or antigen-binding fragment is a monoclonal antibody. In some embodiments, the antibody or antigen-binding fragment is a human antibody. In some embodiments, the antibody or antigen-binding fragment is a IgG1, IgG2, IgG3, or IgG4 class. In some embodiments, the antibody or antigen-binding fragment is a Fab fragment, Fv fragment, or single-chain Fv antibody. In some embodiments, the antibody or antigen-binding fragment comprises vhCDR1, vhCDR2, vhCDR3, vlCDR1, vlCDR2 and vlCDR3 sequences capable of specifically recognizing and binding to one or more epitopes on the SARS-COV-2 nucleocapsid protein.

In some embodiments, provided herein are cell lines that expresses an antibody or antigen-binding fragment capable of binding to one or more epitopes on a SARS-COV-2 nucleocapsid protein and preventing, reducing the likelihood, and/or reducing the severity or length of a SARS-COV-2 infection when administered to a subject.

In some embodiments, provided herein are nucleic acids and expression vectors that encode an antibody or antigen-binding fragment capable of binding to one or more epitopes on a SARS-COV-2 nucleocapsid protein and preventing, reducing the likelihood, and/or reducing the severity or length of a SARS-COV-2 infection when administered to a subject.

In some embodiments, provided herein are methods of treating, preventing, or reducing the severity of a SARS-COV-2 infection in a subject comprising administering a pharmaceutical composition comprising administering an antibody or antigen-binding fragment capable of binding to one or more epitopes on a SARS-COV-2 nucleocapsid protein and preventing, reducing the likelihood, and/or reducing the severity or length of a SARS-COV-2 infection when administered to a subject. In some embodiments, the subject is infected with SARS-COV-2. In some embodiments, the subject exhibits symptoms of SARS-COV-2 infection. In some embodiments, the subject is at risk of infection with SARS-COV-2. In some embodiments, the antibody or antigen-binding fragment is co-administered with one or more additional therapeutic or prophylactic agents.

In some embodiments, provided herein are methods of improving viral clearance of SARS-COV-2 from a subject comprising vaccinating the subject against a SARS-COV-2 nucleocapsid protein or an antigen or epitope thereof. In some embodiments, the subject is not infected with SARS-COV-2. In some embodiments, the vaccination improves the rate of viral clearance for future SARS-COV-2 infections for the subject.

In some embodiments, provided herein are methods of improving viral clearance of SARS-COV-2 from a subject comprising administering a composition comprising antibodies against a SARS-COV-2 nucleocapsid protein or an antigen or epitope thereof. In some embodiments, the composition comprises a pharmaceutical antibody formulation. In some embodiments, the composition comprises a serum or blood product. In some embodiments, the subject is not infected with SARS-COV-2 and the composition is administered prophylactically to improve the rate of viral clearance for future SARS-COV-2 infections for the subject. In some embodiments, the subject is infected with SARS-COV-2 and the composition is administered therapeutically to improve the rate of viral clearance for the SARS-COV-2 infection.

### BRIEF DESCRIPTION OF THE DRAWINGS

FIG. 1A-C. SARS-COV-2 nucleocapsid specific antibody after SARS-COV-2 infection in a cohort of patients admitted to Northwestern University Hospital. (FIG. 1A) Human pre-2019 plasma from healthy individuals were used as control. Data shown are from an ongoing study, in which participants were infected on different dates, hence the heterogeneity in the nucleocapsid-specific antibody responses. SARS-COV-2 infection was confirmed by RT-PCR. Antibody responses were evaluated by ELISA. (FIG. 1B) Summary of SARS-COV-2 nucleocapsid-specific antibodies in sera. (FIG. 1C) Summary of influenza HA-specific antibodies in sera (used as irrelevant antigen control). Dashed lines represent the LOD. Indicated P-value was calculated using Mann Whitney test. Error bars represent SEM.

FIG. 2A-D. A nucleocapsid vaccine improves the control of SARS-COV-2 infection. (FIG. 2A) Representative FACS plots showing the frequencies of SARS-COV-2 nucleocapsid-specific CD8 T cells (D^{b}N219+) in PBMCs. (FIG. 2B) Summary of SARS-COV-2 nucleocapsid-specific antibodies in sera. (FIG. 2C) Weight loss. (D) Viral loads in lungs at day 5 post-infection. K18-hACE2 mice were primed intramuscularly with Ad5 expressing nucleocapsid (Ad5-N), and after 2 weeks, adaptive immune responses were evaluated, and mice were then challenged intranasally with 5×10⁴ PFU of SARS-COV-2. Weight loss was recorded daily, and RNA was harvested from the lungs at day 5 post-infection. Viral RNA was quantified by RT-qPCR. Challenges were performed with a total of 5 mice per group in Biosafety level 3 (BSL-3) facilities. Indicated P-values were calculated using Mann Whitney test.

FIG. 3A-D. Immunogenicity of a SARS-COV-2 nucleocapsid vaccine regimen. (FIG. 3A) Experimental approach for evaluating immune responses after nucleocapsid vaccination. (FIG. 3B) Representative FACS plots showing the frequencies of SARS-COV-2 nucleocapsid-specific CD8 T cells (D^{b}N219+) in PBMCs. (FIG. 3C) Representative FACS plots showing the frequencies of SARS-COV-2 nucleocapsid-specific memory B cells in spleen. Splenocytes were MACS-purified by negative selection to enrich for B cells, facilitating visualization of nucleocapsid-specific B cells. (FIG. 3D) Summary of SARS-COV-2 nucleocapsid-specific antibodies in sera. Data are from week 2 post-boost, and from an experiment with n=5-6 per group. Experiment was performed twice with similar results. Indicated P-values were calculated using Mann Whitney test.

FIG. 4A-C. Nucleocapsid-specific humoral responses do not prevent SARS-COV-2 infection. (FIG. 4A) Experimental approach for performing focus reduction neutralization titer (FRNT) assays on Vero cells using live SARS-COV-2 (USA-WA1/2020). See Materials and Methods for technical information. (FIG. 4B) Representative wells showing the frequencies of SARS-COV-2+ cells (1:450 sera dilution). (FIG. 4C) Summary of FRNT₅₀ titers in sera. Data are from week 3 post-vaccination. Data are from an experiment with n=5 per group. Experiment was performed twice with similar results. Indicated P-values were calculated using Kruskal-Wallis test (multiple comparisons).

FIG. 5A-D. Nucleocapsid-specific humoral responses improve the control of SARS-CoV-2 infection. (FIG. 5A) Experimental approach for evaluating viral control after passive immunization with nucleocapsid-specific sera. (FIG. 5B) Weight loss. (FIG. 5C) Viral loads in lungs by RT-pPCR. (FIG. 5D) Viral loads in lungs by focus forming assays. C57BL/6 mice were primed intramuscularly with Ad5 expressing nucleocapsid (Ad5-N), and after 3 weeks they were boosted with soluble nucleocapsid (N) protein. After 2 weeks post-boost, sera from these mice were pooled and 500 μL of these sera were adoptively transferred into naïve K18-hACE2 recipient mice. On the following day, the K18-hACE2 mice were challenged intranasally with 10³ PFU of SARS-COV-2. RNA was harvested from the lungs at day 4 post-infection, and viral RNA was quantified. Challenges were performed with a total of 4 mice per group in Biosafety level 3 (BSL-3) facilities. Indicated P-values were calculated using Mann Whitney test.

FIG. 6A-I. Nucleocapsid-specific monoclonal antibody (mAb) improves the control of SARS-COV-2 infection. (FIG. 6A) Experimental approach for evaluating viral control after treatment with nucleocapsid-specific mAb during a low dose viral challenge. 800 μg of mAb (IgG control or anti-N) were injected intraperitoneally into naïve K18-hACE2 recipient mice. On the following day, the K18-hACE2 mice were challenged intranasally with 10³ PFU of SARS-CoV-2. (FIG. 6B) Weight loss. (FIG. 6C) Viral loads in lungs by RT-qPCR. RNA was harvested from the lungs at day 7 post-infection, and viral RNA was quantified. (FIG. 6D) Experimental approach for evaluating viral control after treatment with nucleocapsid-specific mAb during a high dose viral challenge. 800 μg of mAb (IgG control or anti-N) were injected intraperitoneally into naïve K18-hACE2 recipient mice. On the following day, the K18-hACE2 mice were challenged intranasally with 5×10⁴ PFU of SARS-COV-2. (FIG. 6E) Weight loss. (FIG. 6F) Viral loads in lungs by RT-qPCR. RNA was harvested from the lungs at day 5 post-infection, and viral RNA was quantified. (FIG. 6G) IL-6 levels in sera. (FIGS. 6H-I) H&E stains of lung. Panels A-C are from low dose viral challenges; panels D-I are from high dose viral challenges. Challenges were performed with a total of 4-5 mice per group in Biosafety level 3 (BSL-3) facilities. Indicated P-values were calculated using Mann Whitney test.

FIG. 7A-B. N-specific antibodies bind to infected cells and trigger ADCC. (FIG. 7A) Representative FACS plots showing that N-specific sera and n-specific mAb bind to SARS-CoV-2 infected cells. 293-ACE2 cells were infected with SARS-COV-2 and binding to N-specific antibody was assessed by flow cytometry using a secondary antibody bound to Alexa 647. Control cells were mock infected. (FIG. 7B) N-specific antibodies trigger ADCC. 293-ACE2 cells were infected with SARS-COV-2 and co-cultured with effectors (NKL-mCD16 cells), together with N-specific immune sera or anti-N mAb (clone 1C7). CD107a expression on NKL-mCD16 cells was quantified by flow cytometry after 5 hr. Data are from 1:30 dilution. Data are from two experiments, each with n=5 per group. All data are shown. Indicated P-values were calculated using two-way ANOVA test (Dunnett's multiple comparisons test).

FIG. 8. Nucleocapsid-specific monoclonal antibody (mAb) improves the control of SARS-COV-2 infection. Viral loads in lungs by focus forming assays. These data are from the same mice from FIG. 6A-6C, except that viral loads in lungs were quantified by focus forming assays. Challenges were performed with a total of 4-5 mice per group in Biosafety level 3 (BSL-3) facilities. Indicated P-values were calculated using Mann Whitney test.

FIG. 9. Nucleocapsid-specific monoclonal antibody (mAb) improves the acute control of SARS-COV-2 infection. Viral loads in lungs by RT-qPCR. Experiment was identical to that of FIG. 6A, except that mice were euthanized after 4 days. Challenges were performed with a total of 4-5 mice per group in Biosafety level 3 (BSL-3) facilities. Indicated P-values were calculated using Mann Whitney test.

FIG. 10A-B. Nucleocapsid-specific monoclonal antibody (mAb) does not significantly affect virus-specific CD8 T cell responses. (FIG. 10A) Numbers of SARS-COV-2 nucleocapsid-specific CD8 T cells (D^{b}N219+). (FIG. 10B) Numbers of SARS-COV-2 spike-specific CD8 T cells (D^{b}N219+). These data are from the same mice from FIG. 6A-6C. Data are from spleen at day 7 post-infection. Challenges were performed with a total of 4-5 mice per group in Biosafety level 3 (BSL-3) facilities. Indicated P-values were calculated using Mann Whitney test.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an antibody” is a reference to one or more antibodies and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “preventing” refers to prophylactic steps taken to reduce the likelihood of a subject (e.g., an at-risk subject) from developing or suffering from a particular disease, disorder, or condition (e.g., asthma). The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., intravenously, subcutaneously, etc.), orally, etc.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

The term “amino acid” refers to natural amino acids, unnatural amino acids, and amino acid analogs, all in their D and L stereoisomers, unless otherwise indicated, if their structures allow such stercoisomeric forms. Embodiments herein refer to various amino acid abbreviations (single-letter or three-letter abbreviations) that will be understood by those in the field. Any amino acid abbreviations not defined herein refer to their field-accepted meaning. For example, “NMe” preceding an amino acid name refers to an “N-methyl” group on the amino acid, “Nle” is “norleucine,” “Abu” is “α-Aminobutyric acid,” “Aib” is “2-Aminoisobutyric acid,” “Nal(2′) is “3-(2-Naphthyl)-L-alanine,” “tic” is “1,2,3,4-tetrahydroisoquinoline-3-carboxylic acid,” “HpH” is “homophenylalanine,” “Bip” is “N-alpha-Fmoc-beta-(4-biphenyl)-L-alanine,” “D-Phc(4tBu)” is “D-4-tert-butyl-phenylalanine,” and the single-letter or three-letter abbreviations for the common proteinogenic amino acids are provided below.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyroolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 30 amino acids or less in length. A peptide may comprise natural amino acids, non-natural amino acids, proteinogenic amino acids, non-proteinogenic amino acids, amino acid analogs, and/or modified amino acids. A peptide may be a subsequence of naturally occurring protein or a non-natural (artificial) sequence.

As used herein, the term “artificial” refers to compositions and systems that are designed or prepared synthetically, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid I;
3) Asparagine (N) and Glutamine (Q);
4) Arginine I and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine I and Methionine (M).

Naturally occurring residues may be divided into classes based on common side chain properties, for example: polar positive (or basic) (histidine (H), lysine (K), and arginine I); polar negative (or acidic) (aspartic acid (D), glutamic acid I); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 20 amino acids) may have up to X substitutions (e.g., 2) relative to SEQ ID NO:Z, and may therefore also be expressed as “having X (e.g., 2) or fewer substitutions relative to SEQ ID NO:Z.”

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (β2M), CD3, CD4 and CD8. Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, β, γ, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; a and y and comprise approximately 450 amino acids, while u has approximately 550 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (about 66%), followed by IgG2 (about 23%), IgG3 (about 7%) and IgG4 (about 4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437).

Antibodies are not only found as monomers but are also known to form dimers of two Ig units (e.g. IgA), tetramers of four Ig units (e.g. IgM of telcost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel β-strands arranged in two-sheets. Typically, the “heavy chain” of an antibody comprises four Ig domains with three of them being constant (CH domains: CH1, CH2, CH3) domains and one of the being a variable domain (V), with the exception of IgM and IgE which contain one variable (VH) and four constant regions (CH1, CH2, CH3, CH4). The additional domain (CH2: Cμ2, C∈2) in the heavy chains of IgM and IgE molecules connects the two heavy chains instead of the hinge region contained in other Ig molecules (Perkins et al., (1991) J Mol Biol. 221(4): 1345-66; Beavil et al., (1995) Biochem 34(44): 14449-61; Wan et al., (2002) Nat Immunol. 3(7):681-6). The “light chain” typically comprises one constant Ig domain (CL) and one variable Ig domain (VL). Exemplified, the human IgM heavy chain is composed of four Ig domains linked from N- to C-terminus in the order VH-CH1-CH2-CH3-CH4 (also referred to as VH-Cμ1-Cμ2-Cμ3-Cμ4), whereas the human IgM light chain is composed of two immunoglobulin domains linked from N- to C-terminus in the order VL-CL, being either of the kappa or lambda type (Vκ-Cκ or Vλ-Cλ).

Exemplified, the constant chain of human IgM comprises 452 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5th ed. U.S. Department of Health and Human Service, National Institutes of Health, Bethesda, Md. The “EU index as in Kabat” refers to the residue numbering of the human IgM EU antibody. Accordingly, CH domains in the context of IgM are as follows: “CH1” refers to amino acid positions 118-215 according to the EU index as in Kabat; “CH2” refers to amino acid positions 231-340 according to the EU index as in Kabat; “CH3” refers to amino acid positions 341-446 according to the EU index as in Kabat. “CH4” refers to amino acid positions 447-558 according to the OU index as in Kabat.

Whilst in human IgA, IgG, and IgD molecules two heavy chains are connected via their hinge region, IgE and IgM antibodies do not comprise such hinge region. Instead, IgE and IgM antibodies possess an additional Ig domain, their CH2 domain, which functions as dimerization domain between two heavy chains. In contrast to rather flexible and linear hinge regions of other antibodies, the CH2 domain of IgE and IgM are composed of two beta sheets stabilized by an intradomain disulfide bond forming a c-type immunoglobulin fold (Bork et al., (1994) J Mol Biol. 242(4):309-20; Wan et al., (2002) Nat Immunol. 3(7):681-6). Furthermore, the MHD2 and EHD2 domains contain one N-glycosylation site.

The “IgM heavy chain domain 2” (“MHD2”) consists of 111 amino acid residues (12.2 kDa) forming a homodimer covalently held together by a disulfide bond formed between cysteine residue 337 of two domains (Davis et al., (1989) EMBO J 8(9):2519-26; Davis & Shulman, (1989) Immunol Today. 10(4):118-22; 127-8). The domain is further stabilized by an intradomain disulfide bond formed between Cys261 and Cys321. Typically, two MHD2 domains are covalently linked by an interdomain disulfide bond between Cys337. The MHD2 contains an N-glycosylation site at Asn333.

“Fc” or “Fc region” or “Fc domain” as used herein refers to the polypeptide comprising the constant region of an antibody excluding the first constant region immunoglobulin domain and, in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may or may not include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, as is more fully described below, amino acid modifications are made to the Fc region, for example to alter binding to one or more FcγR receptors or to the FcRn receptor.

As used herein, the term “antibody” refers to a whole antibody molecule or a fragment thereof (e.g., fragments such as Fab, Fab′, and F(ab′)2), it may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, VH, and three constant regions, C_{H1}, C_{H2}, and C_{H3}. The V_{H} domain is at the amino-terminus of the heavy chain, and the CH3 domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. Sec, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. Sec, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. Sec, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

As used herein, the term “human antibody” means any antibody in which the variable and constant domain sequences are human sequences. The term encompasses antibodies acquired from and/or enriched from a human sourced starting material, e.g., plasma from a recovered donor infected with SARS-COV-2.

A “neutralizing antibody”, an antibody with “neutralizing activity”, “antagonistic antibody”, or “inhibitory antibody”, as used herein, means an antibody capable of preventing, retarding or diminishing replication of the viral target of the antibody. In some embodiments, neutralizing antibodies are effective at antibody concentrations of <0.2 μg/mL. In some embodiments, neutralizing antibodies are effective at antibody concentrations of <0.1 μg/mL.

The term “vaccination” or “vaccinate” means administration of a vaccine that can elicit an immune response or confer immunity from a disease.

The term “adjuvant” refers to agents that augment, stimulate, activate, potentiate, or modulate the immune response to the active ingredient of the composition at either the cellular or humoral level, e.g. immunologic adjuvants stimulate the response of the immune system to the actual antigen, but have no immunological effect themselves. Adjuvants are used to accomplish three objectives: (1) they slow the release of antigens from the injection site; (2) they stimulate the immune system; and (3) the addition of an adjuvant may permit the use of a smaller dose of antigen to stimulate a similar immune response, thereby reducing the production cost of the vaccine. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-113, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g. polyarginine or polylysine).

The term “coronavirus” or “CoV” refers to any virus of the coronavirus family, including but not limited to SARS-COV-2, MERS-COV, and SARS-COV-1. “SARS-COV-2” refers to the coronavirus responsible for causing COVID-19 disease and symptoms related thereto.

The term “coronavirus infection”, “SARS-COV-2 infection”, or “CoV infection,” as used herein, refers to infection with a coronavirus such as SARS-COV-2. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

As used herein, a human subject is “symptomatic” of a SARS-COV-2 infection if the subject shows one or more symptoms known to appear in a SARS-COV-2-infected human subject after a suitable incubation period. Such symptoms include, without limitation, detectable SARS-COV-2 in the subject, and those symptoms shown by patients afflicted with SARS-COV-2. SARS-COV-2-related symptoms include, without limitation, respiratory distress, hypoxia, difficulty breathing (dyspnea), cardiovascular collapse, arrhythmia (e.g., atrial fibrillation, tachycardia, bradycardia), fatigue, altered mental status (including confusion), cough, fever, chills, abnormal blood coagulation events, myalgia, loss of smell and/ortaste, loss of appetite, nausea, red/watery eyes, dizziness, stomach-ache, rash, sneezing, sputum/phlegm, and runny nose.

The term “CoV-N”, also called “N protein”, refers to the nucleocapsid protein of SARS-CoV-2. The N protein is highly abundant in SARS-COV-2 virions and infected cells. The N protein plays several roles in the SARS-COV-2 life cycle, including facilitating viral RNA production, suppressing host cells' innate immune responses, and packaging viral genomic RNA into developing virions. The N protein has an amino acid sequence of SEQ ID NO: 1 or variants thereof. The N protein possesses a modular structure with an N-terminal RNA-binding domain (RBD) and a C-terminal dimerization domain (CTD), plus three intrinsically disordered regions (IDRs) at the N- and C-termini and between the RBD and CTD. The protein oligomerizes through its CTD and disordered C-terminal tail, and the protein also undergoes liquid-liquid phase separation with RNA mediated by its RBD and central disordered region. In cells, N protein condensates recruit the stress granule proteins G3BP1 and G3BP2, suppressing stress granule assembly. During virion production, the N protein assembles into viral RNA-protein complexes (RNPs) with a characteristic barrel shape to package the viral RNA, and interacts with the Membrane protein to recruit the viral genome to developing virions

The term “CoV-S”, also called “S protein”, refers to the spike protein of SARS-COV-2.

### DETAILED DESCRIPTION

Provided herein are compositions and methods for the treatment and/or prevention of SARS-COV-2 infection, and symptoms and conditions associated therewith (e.g., COVID-19). In particular, provided herein are nucleocapsid-specific antibodies and methods of use thereof therapeutically or prophylactically to enhance the clearance of SARS-COV-2 infection from a subject.

The SARS-COV-2 spike protein is the main antigen in all approved COVID-19 vaccines and is also the only current target for monoclonal antibody therapies. Immune responses to other viral antigens are generated after SARS-COV-2 infection, but their contribution to the antiviral response remains unclear. Experiments were conducted during development of embodiments herein to interrogate whether nucleocapsid-specific antibodies can improve protection against SARS-COV-2. Mice were immunized with a nucleocapsid-based vaccine, and then transferred sera from these mice into naïve mice, followed by challenge with SARS-COV-2. The experiments demonstrate that the mice that received nucleocapsid-specific sera or a nucleocapsid-specific monoclonal antibody (mAb) exhibited enhanced control of SARS-COV-2. Nucleocapsid-specific antibodies elicited NK-mediated antibody-dependent cellular cytotoxicity (ADCC) against infected cells. The nucleocapsid-specific humoral responses and a nucleocapsid-specific monoclonal antibody mediate antibody-dependent cellular cytotoxicity (ADCC) and help control SARS-COV-2 infection when given as pre-exposure prophylaxis.

The SARS-COV-2 spike protein mediates viral entry by binding to the ACE2 receptor, and therefore, this protein is considered the most important antigenic target for vaccines and monoclonal antibody therapies. All approved SARS-COV-2 vaccines target the spike protein with the goal of generating spike-specific antibodies that block viral entry. However, it is unclear if antibodies of other specificities (e.g. internal viral proteins that do not mediate viral entry) can play a role in antiviral protection. In particular, nucleocapsid-specific antibodies are generated after SARS-COV-2 infection and also after immunization with experimental nucleocapsid-based vaccines (14, 22), but it is unclear if nucleocapsid-specific antibody responses can confer any protection in vivo.

Experiments conducted during development of embodiments herein provide the first demonstration that nucleocapsid-specific humoral responses assists in clearing a SARS-COV-2 infection. Current monoclonal antibody therapies for COVID-19 target only the spike protein, and many of these therapies have lost efficacy against variants, since the spike protein is highly variable. The vast majority of mutations in SARS-COV-2 are focused in the spike. Similarly, most of the genetic diversity in human immunodeficiency virus (HIV) (and other rapidly mutating viruses) occurs in the envelope proteins that mediate viral entry, motivating the development of mAb therapies targeting exclusively these surface viral proteins for prevention and treatment of HIV infection (Refs. 26, 27; incorporated by reference in their entireties). Nucleocapsid-specific antibodies control infection. Following initial infection, virus can transmit via cell-to-cell interactions and this type of infection is resistant to neutralizing antibodies, but not antibody effector mechanisms that target cell-surface antigens (Ref. 28; incorporated by reference in its entirety).

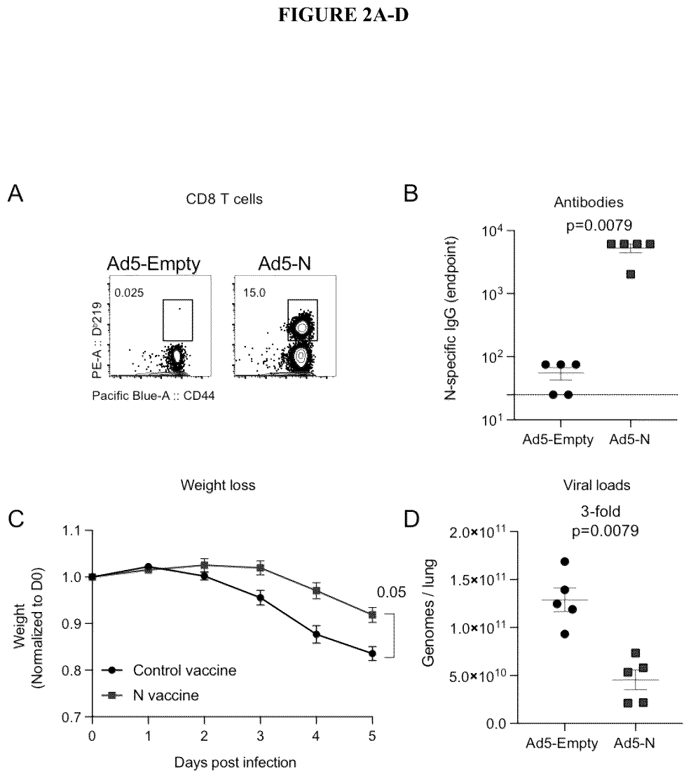

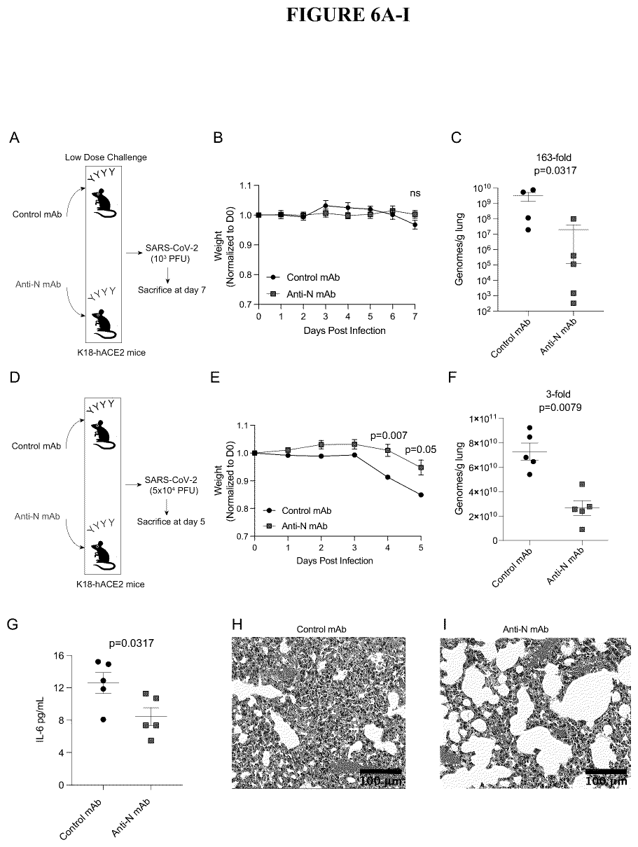

Previous experiments had indicated that a nucleocapsid-based vaccine conferred limited protection against a SARS-COV-2 challenge when given as a “single vaccine” without a spike-based vaccine. In that prior study, viral loads were evaluated during the hyperacute phase of infection (72 hr), which may have been too early to observe virology differences, given that the efficacy of nucleocapsid-specific antibodies may rely on the recruitment and effector mechanisms of immune cells. In experiments conducted during development of embodiments herein, following low dose viral challenges (10³ PFU), which represent viral loads that humans typically encounter during a natural infection, nucleocapsid-specific antibodies elicited a 163-fold reduction in viral titers (FIG. 6C). However, following high dose viral challenges (5×10⁴ PFU), nucleocapsid-specific antibodies elicited only a 3-fold reduction in viral titers (FIGS. 2D and 6F). Although the antiviral effect was more limited during the high dose viral challenge, there was an overall reduction in emaciation and inflammation, following treatment with nucleocapsid-specific antibodies.

In some embodiments, provided herein are anti-CoV-N antibodies and pharmaceutical compositions comprising anti-CoV-N antibodies. The antibodies of the invention are specific for the nucleocapsid protein of SARS-COV-2. In some embodiments, provided herein are methods of treating a SARS-COV-2 in a subject (e.g., an infected subject, a symptomatic subject, a hospitalized subject, etc.) comprising administering a pharmaceutical composition and/or immunotherapy comprising anti-CoV-N antibodies. In some embodiments, provided herein are methods of preventing a SARS-COV-2 infection or symptoms or diseases (e.g., COVID-19) related thereto comprising administering a pharmaceutical composition and/or immunotherapy comprising anti-CoV-N antibodies to a subject. In some embodiments, provided herein are methods of reducing the likelihood of a subject becoming infected with SARS-COV-2 and/or developing symptoms or diseases (e.g., COVID-19) related thereto comprising administering a pharmaceutical composition and/or immunotherapy comprising anti-CoV-N antibodies to a subject.

In an exemplary embodiment, administration of a pharmaceutical composition and/or immunotherapy comprising anti-CoV-N antibodies reduces the risk of mortality of the subject. In some embodiments, the administration results in shortened time of recovery. In one embodiment, the subject is monitored using RNA PCR to test for lower or negative viral titer in total lung tissue and/or sputum.

The present technology provides anti-CoV-N antibodies. The antibodies of the invention are specific for the nucleocapsid of SARS-COV-2 as more fully outlined herein and below. In some embodiments, methods are provided of administering anti-CoV-N antibodies to a subject for therapeutic and/or prophylactic purposes.

In some embodiments, anti-CoV-N antibodies of the compositions and methods described herein bind to one or more epitopes on the nucleocapsid protein of SARS-COV-2. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”.

Included within the definition of “antibody” herein is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to CoV-N antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site.

An antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques

The present invention provides a pharmaceutical compositions comprising an anti-CoV-N antibody or antigen-binding fragment thereof, and methods of use thereof. In some embodiments, anti-CoV-N antibodies or antigen-binding fragments thereof comprise variable regions (e.g., heavy and/or light chains) capable of specifically binding to one or more epitopes displayed by the SARS-COV-2 nucleocapsid protein. In some embodiments, anti-CoV-N antibodies or antigen-binding fragments thereof comprise complementarity determining regions (CDRs) capable of recognizing and binding to one or more specific epitopes displayed by the SARS-COV-2 nucleocapsid protein (e.g., HCDR1, HCDR2, HCDR3, LCDR1, LCDR2, LCDR3).

The therapeutic compositions used in the practice of the present invention can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibodies of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Exemplary ones are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

Administration of the pharmaceutical composition comprising antibodies of the present invention, for example in the form of a sterile aqueous solution, may be done in a variety of ways, including, but not limited to subcutaneously, intravenously, and intranasally. Subcutaneous administration may be done in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO04091658). Fc polypeptides of the present invention may be more amenable to subcutaneous administration due to, for example, increased potency, improved serum half-life, or enhanced solubility.

As is known in the art, protein therapeutics are often delivered by IV infusion or bolus. The antibodies of the present invention may also be delivered using such methods. For example, administration may be by intravenous infusion with 0.9% sodium chloride as an infusion vehicle.

In addition, any of a number of delivery systems are known in the art and may be used to administer the Fc variants of the present invention. Examples include, but are not limited to, encapsulation in liposomes, microparticles, microspheres (eg. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT®, and poly-D-(−)-3-hydroxyburyric acid. The antibodies disclosed herein may also be formulated as immunoliposomes. A liposome is a small vesicle comprising various types of lipids, phospholipids and/or surfactant that is useful for delivery of a therapeutic agent to a mammal. Liposomes containing the antibody are prepared by methods known in the art, such as described in Epstein et al., 1985, Proc Natl Acad Sci USA, 82:3688; Hwang et al., 1980, Proc Natl Acad Sci USA, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The antibodies may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT® (which are injectable microspheres composed of lactic acid-glycolic acid copolymer and leuprolide acetate), poly-D-(−)-3-hydroxybutyric acid, and Protease® (commercially available from Alkermes), which is a microsphere-based delivery system composed of the desired bioactive molecule incorporated into a matrix of poly-DL-lactide-co-glycolide (PLG).

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the antibody in the formulation may vary from about 0.1 to 100 weight %. In some embodiments, the concentration of the Fc variant is in the range of 0.003 to 1.0 molar. In order to treat a patient, a therapeutically effective dose of the Fc variant of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from about 0.0001 to 100 mg/kg of body weight or greater, for example about 0.1, 1, 10, or 50 mg/kg of body weight, and in an exemplary embodiment, from about 1 to 10 mg/kg.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the present invention provides nucleic acids encoding the CoV-N antibodies or CoV-N-binding domains of. As will be appreciated by those in the art, the protein sequences depicted herein can be encoded by any number of possible nucleic acid sequences, due to the degeneracy of the genetic code. In some embodiments, the nucleic acid molecules are DNA. In some embodiments, the nucleic acid molecules are RNA.

Provided herein are methods for treating or preventing viral infection (e.g., coronavirus infection) by administering a therapeutically (or prophylactically) effective amount of anti-CoV-N antigen-binding protein, e.g., antibody or antigen-binding fragment, to a subject (e.g., a human) in need of such treatment or prevention. In some embodiments, SARS-COV-2 infection may be treated or prevented, in a subject, by administering an antibody or antigen-binding fragment of the present invention to a subject. In some embodiments, COVID-19 is treated or prevented, in a subject, by administering an antibody or antigen-binding fragment of the present invention to a subject. In some embodiments, symptoms of SARS-COV-2 infection are treated or prevented, in a subject, by administering an antibody or antigen-binding fragment of the present invention to a subject. In some embodiments, methods herein do not prevent infection of a subject by SARS-COV-2, but are effective in treating/preventing symptoms of the infection and/or promoting/accelerating recovery.

An effective or therapeutically effective dose of anti-CoV-N antigen-binding protein, e.g., antibody or antigen-binding fragment, for treating or preventing a viral infection refers to the amount of the antibody or fragment sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the invention, an effective or therapeutically effective dose of antibody or antigen-binding fragment thereof of the present invention, for treating or preventing viral infection, e.g., in an adult human subject, is about 0.01 to about 200 mg/kg, e.g., up to about 150 mg/kg. Depending on the severity of the infection, the frequency and the duration of the treatment can be adjusted. In certain embodiments, the antigen-binding protein of the present invention can be administered at an initial dose, followed by one or more secondary doses. In certain embodiments, the initial dose may be followed by administration of a second or a plurality of subsequent doses of antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

In some embodiments, the method of preventing viral infection provided herein comprises prophylactically administering an antibody or antigen-binding fragment of the present invention, to a subject who is at risk of viral infection so as to prevent such infection. Passive antibody-based immunoprophylaxis has proven an effective strategy for preventing subject from viral infection.

In an embodiment of the invention, a sign or symptom of a viral infection in a subject is survival or proliferation of virus in the body of the subject, e.g., as determined by viral titer assay (e.g., coronavirus propagation in embryonated chicken eggs or coronavirus protein assay). Other signs and symptoms of viral infection are discussed herein.

In some embodiments, provided herein are methods for treating or preventing viral infection (e.g., coronavirus infection) or for inducing the regression or elimination or inhibiting the progression of at least one sign or symptom of viral infection such as: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection, in a subject in need thereof (e.g., a human), by administering a therapeutically effective amount of antibody or antigen-binding fragment to the subject, for example, by injection of the protein into the body of the subject. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a SARS-COV-2 infection (i.e., in a statistically significant manner). In certain embodiments, therapeutic or prophylactic/preventive benefit includes a reduced duration of hospitalization for treatment of a SARS-COV-2 infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A number of criteria are believed to contribute to high risk for severe symptoms or death associated with a SARS COV-2 infection. These include, but are not limited to, age, occupation, general health, pre-existing health conditions, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are believed to be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a human subject treated according to embodiments herein is a resident of a nursing home or a long-term care facility, is a hospice care worker, is a healthcare provider or healthcare worker, is a first responder, is a family member or other close contact of a subject diagnosed with or suspected of having a SARS-COV-2 infection, is overweight or clinically obese, is or has been a smoker, has or had chronic obstructive pulmonary disease (COPD), is asthmatic (e.g., having moderate to severe asthma), has an autoimmune disease or condition (e.g., diabetes), and/or has a compromised or depleted immune system (e.g., due to AIDS/HIV infection, a cancer such as a blood cancer, a lymphodepleting therapy such as a chemotherapy, a bone marrow or organ transplantation, or a genetic immune condition), has chronic liver disease, has cardiovascular disease, has a pulmonary or heart defect, works or otherwise spends time in close proximity with others, such as in a factory, shipping center, hospital setting, or the like.

In certain embodiments, a subject treated according to the present disclosure has received a vaccine for SARS-COV-2 and the vaccine is determined to be ineffective, e.g., by post-vaccine infection or symptoms in the subject, by clinical diagnosis or scientific or regulatory criteria.

In certain embodiments, treatment is administered as peri-exposure prophylaxis. In certain embodiments, treatment is administered to a subject with mild-to-moderate disease, which may be in an outpatient setting. In certain embodiments, treatment is administered to a subject with moderate-to-severe disease, such as requiring hospitalization.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

In some embodiments, provided herein are methods of co-administering an anti-CoV-N antigen-binding protein, e.g., antibody or antigen-binding fragment, with one or more additional therapeutics. In some embodiments, the additional therapeutic(s) and the anti-CoV-N antigen-binding protein are administered in parallel (e.g., simultaneously). In some embodiments, the additional therapeutic(s) and the anti-CoV-N antigen-binding protein are present in a single pharmaceutical composition or formulation. In some embodiments, provided herein are compositions comprising an additional therapeutic and an anti-CoV-N antigen-binding protein. In some embodiments, the additional therapeutic(s) and the anti-CoV-N antigen-binding protein are provided as separate pharmaceutical compositions or formulations. In some embodiments, the additional therapeutic(s) and the anti-CoV-N antigen-binding protein are administered in series (e.g., at distinct time points separated by 1 hour, 2 hours, 4 hours, 8 hours, 12 hours, 1 day, 2 days, 4 days, 1 week, 2 weeks, one month, or more). In some embodiments, suitable additional therapeutics comprises at least one other therapeutic, prophylactic and/or diagnostic agent. Preferably, the therapeutic and/or prophylactic agents are capable of preventing and/or treating a coronavirus infection and/or a condition/symptom resulting from such an infection. Therapeutic and/or prophylactic agents include, but are not limited to, antiviral agents. Such agents can be binding molecules, small molecules, organic or inorganic compounds, enzymes, polynucleotide sequences, antiviral peptides, etc. The therapeutic and/or prophylactic agent can comprise an M2 inhibitor (e.g., amantadine, rimantadine) and/or a neuraminidase inhibitor (e.g., zanamivir, oseltamivir). In various embodiments, the anti-viral agent can comprise baloxavir, oseltamivir, zanamivir, peramivir, remdesivir, or any combination thereof. The therapeutic and/or prophylactic agent can also include various anti-malarial such as chloroquine, hydroxychloroquine, and analogues thereof.

### EXPERIMENTAL

### Methods

### Cell Lines

Adenoviral vectors were propagated using HEK293 cells purchased from ATCC (cat #CRL-1573). Vero E6 cells were used to propagate SARS-COV-2 isolate USA-WA1/2020 (BEI resources, NR-52281).

### Mice and Vaccinations

6-8-week-old mice were used in these studies. Wild type C57BL/6 mice were used for immunogenicity studies, and K18-hACE2 mice (on C57BL/6 background) were used for challenge studies. These mice express the human ACE2 protein behind the keratin 18 promoter, directing expression in epithelial cells. K18-hACE2 mice were purchased from Jackson laboratories (Stock No: 034860). Mice were immunized intramuscularly (50 μL per quadriceps) with an Ad5 vector expressing SARS-COV-2 nucleocapsid protein (Ad5-N) at 10¹¹ PFU per mouse, and N protein; diluted in sterile PBS. Ad5-N was a kind gift of the Masopust/Vezys laboratory (21). This is a non-replicating Ad5 vector (E1/E3 deleted). The vector contains a CMV (Cytomegalovirus) promoter driving the expression of the respective proteins. The Ad5 vector was propagated on trans-complementing HEK293 cells (ATCC), purified by cesium chloride density gradient centrifugation, titrated, and then frozen at −80° C.

### SARS-COV-2 Virus and Infections

SARS-Related Coronavirus 2, Isolate USA-WA1/2020, NR-52281 was deposited by the Centers for Disease Control and Prevention and obtained through BEI Resources, NIAID, NIH. Virus was propagated and tittered on Vero-E6 cells (ATCC). In brief, Vero cells were passaged in DMEM with 10% Fetal bovine serum (FBS) and Glutamax. Cells less than 20 passages were used for all studies. Virus stocks were expanded in Vero-E6 cells following a low MOI (0.01) inoculation and harvested after 4 days. Viral titers were determined by plaque assay on Vero-E6 cells. Viral stocks were used after a single expansion (passage=1) to prevent genetic drift (a small proportion of the virus stock contained mutations in the furin cleavage site). K18-hACE2 mice were anesthetized with isoflurane and challenged with 10³ PFU of SARS-COV-2 intranasally. Mouse infections were performed at the University of Illinois at Chicago (UIC) following BL3 guidelines with approval by the UIC Institutional Animal Care and Use Committee (IACUC).

### SARS-COV-2 RNA Quantification

Lungs were harvested from infected mice and homogenized in PBS. RNA was isolated with the Zymo 96-well RNA isolation kit (Catalog #: R1052) following the manufacturer's protocol. SARS-COV-2 viral burden was measured by RT-qPCR using Taqman primer and probe sets from IDT with the following sequences: Forward 5′ GAC CCC AAA ATC AGC GAA AT 3′ (SEQ ID NO: 2), Reverse 5′ TCT GGT TAC TGC CAG TTG AAT CTG 3′ (SEQ ID NO: 3), Probe 5′ ACC CCG CAT TAC GTT TGG TGG ACC 3′(SEQ ID NO: 4). A SARS-COV-2 copy number control was obtained from BEI (NR-52358) and used to quantify SARS-COV-2 genomes.

### Focus Forming Assay (FFA) and Focus Reduction Neutralization Titer (FRNT) Assay Using Live SARS-COV-2

Quantification of SARS-COV-2 by FFA was performed by serial dilution of viral stocks or lung homogenate. Dilutions were added onto a monolayer of Vero cells in a 96 well plate. One hour after infection, cells were overlaid with 1% (w/v) methylcellulose in 2% FBS, 1×MEM. Plates were fixed for 30 minutes with 4% PFA 24 hr after infection. Staining involved 1º anti-SARS guinea pig (1:15,000, NR-10361 from BEI Resources) and 2° goat anti-guinea pig HRP (200 ng/ml) in Perm Wash Buffer (0.1% Saponin, 0.1% BSA, in PBS). Treatment with TrueBlue peroxidase substrate (KPL) produced FFU that were quantified with an ImmunoSpot® ELISpot plate scanner (Cellular Technology Limited). For FRNT assays, serial dilutions of heat-inactivated serum from vaccinated mice were incubated with 100 FFU of live SARS-COV-2 (isolate USA-WA1/2020) for one hour at 37° C. before infecting a monolayer of Vero cells in a 96-well plate. Viral foci were determined as above for FFA.

### Reagents, Flow Cytometry and Equipment

Single cell suspensions were obtained from PBMCs or tissues. Dead cells were gated out using Live/Dead fixable dead cell stain (Invitrogen). SARS-COV-2 nucleocapsid protein was biotinylated and conjugated to streptavidin-PE for detection of nucleocapsid-specific memory B cells on MACS-purified B cells. MHC class I monomers (d^{b}219, LALLLLDRL; and K^{b}VL8, VNFNFNGL) were used for detecting virus-specific CD8 T cells, and were obtained from the NIH tetramer facility located at Emory University. MHC monomers were tetramerized in-house. Cells were stained with fluorescently-labeled antibodies against CD8a (53-6.7 on PerCP-Cγ5.5), CD44 (IM7 on Pacific Blue), and K^{b}N219 (PE). Fluorescently-labeled antibodies were purchased from BD Pharmingen, except for anti-CD44 (which was from Biolegend). Flow cytometry samples were acquired with a Becton Dickinson Canto II or an LSRII and analyzed using FlowJo v 10 (Treestar).

### SARS-COV-2 Nucleocapsid Specific ELISA

Binding antibody titers were quantified using ELISA as described previously (Refs. 31, 32; incorporated by reference in their entireties), using nucleocapsid protein as coating antigens. In brief, 96-well flat bottom plates MaxiSorp (Thermo Scientific) were coated with SARS-COV-2 nucleocapsid protein, washed and blocked. Serial sera dilutions were performed. Absorbance was measured using a Spectramax Plus 384 (Molecular Devices). Antibody levels were reported as endpoint titer using serial 3-fold dilutions.

### Sera Transfers and Monoclonal Antibody Therapies

For the passive immunization studies, 500 μL of N-specific immune sera or irrelevant sera were transferred one day before SARS-COV-2 challenge. Control mAb (IgG2a, clone C1.18.4) and Anti-N mAb (clone 1C7C7) were purchased from Leinco. Mice received 800 μg of the respective antibody clone diluted in PBS one day before SARS-COV-2 challenge. Sera transfers and mAb therapy were administered intraperitoneally.

### ADCC Assays

293-ACE2 cells were made by transducing 293 cells with a lentivirus expressing human ACE2, and selected in Blasticidin for 7 days, as previously described (33). Effector NKL cells expressing mouse CD16 (NKL-mCD16) were a kind gift from O.Aguilar and were maintained as previously described (34). In brief, 293-ACE2 cells were mock-infected or infected with SARS-CoV-2 (England2 strain) at MOI=5. After 24 hr, 293-ACE2 cells (25,000/well) were cultured with effectors (NKL-mCD16, 50,000/well), together with sera or mAb (1C7) in the presence of CD107a-FITC (Biolegend) and Golgi-stop (BD Biosciences). 5 h later cultures were washed and stained with live/dead fixable aqua, and CD56-BV605 (Biolegend). CD107a levels were analyzed on an Attune flow cytometer (Thermo Fisher). Cells were gated on the live/CD56+ population, and the percentage of CD107a+ cells was calculated. All sera/mAb samples were incubated with both infected and mock-infected cells to control for non-specific activation. Samples were tested over a 3-fold dilution series beginning at 1:30, to account for “hooking” effects. Every sample was tested in technical duplicate and averaged.

### Detecting Binding of N-Specific Antibodies to Infected Cells

293-ACE2 cells were infected with SARS-COV-2 (England2 strain) at MOI=5. After 24 hr, cells were detached with TrypLE, and stained with primary N-specific mAb (or N-specific immune sera) as indicated, for 30 min at 4° C. After washing, cells were incubated with a secondary antibody (anti-mouse Alexa-Fluor 647) for 30 min at 4° C. Cells were washed, fixed in 4% PFA, and analyzed on an Attune flow cytometer.

### Statistical Analysis

Statistical analyses are indicated on the figure legend. Dashed lines in data figures represent limit of detection. Statistical significance was established at p≤0.05 and was generally assessed by Mann Whitney tests, unless indicated otherwise in figure legends. Data were analyzed using Prism (Graphpad).

### Results

### Adaptive Immune Responses Elicited by a Nucleocapsid Vaccine Help Control a SARS-COV-2 Infection

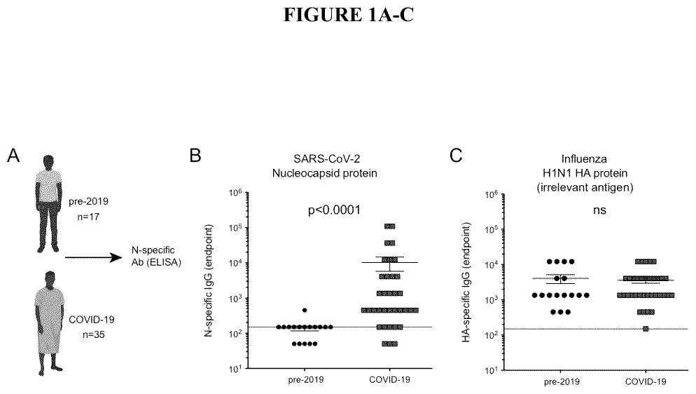

All approved COVID-19 vaccines express the spike protein of SARS-COV-2. Immune responses against other antigens, for example against the nucleocapsid antigen, are not elicited after SARS-COV-2 vaccination, but can be induced after natural SARS-COV-2 infection. As shown in FIG. 1A-1B, nucleocapsid-specific antibody responses were detected in the plasma of COVID-19 patients, but not in the plasma of individuals before the 2019 pandemic. Similar antibody responses were detected to an irrelevant viral antigen (Influenza) in SARS-COV-2 exposed and unexposed individuals (FIG. 1C).

It has been shown that a nucleocapsid-based vaccine does not confer significant protection against an intranasal SARS-COV-2 challenge when given as a “single vaccine,” without a spike-based vaccine (Ref. 14; incorporated by reference in its entirety). Viral loads were evaluated at a very early time post-infection (day 3 post-infection) to measure breakthrough infection. In follow up studies, viral control was evaluated at later times post-infection. K18-hACE2 mice were vaccinated intramuscularly with an adenovirus serotype 5 vector expressing SARS-COV-2 nucleocapsid (Ad5-N) at a dose of 10¹¹ PFU per mouse. K18-hACE2 mice were utilized because they are susceptible to SARS-COV-2 and are widely used to evaluate vaccines (Refs. 14-19; incorporated by reference in their entireties). Two weeks post-vaccination, nucleocapsid-specific CD8 T cell responses (FIG. 2A) and antibody responses (FIG. 2B) in mice vaccinated with the nucleocapsid vaccine were detected. The K18-hACE2 mice were then challenged intranasally with a high dose (4×10⁵ PFU) of SARS-COV-2 (isolate USA-WA1/2020), and at day 5 post-challenge viral loads were quantified in lungs by RT-qPCR. Mice that received the nucleocapsid vaccine showed significantly lower weight loss (FIG. 2C) and lower viral loads (FIG. 2D) compared to control vaccinated mice. Altogether earlier studies had indicated that a nucleocapsid vaccine does not prevent breakthrough infection and does not significantly reduce viral titers during the hyperacute phase (day 3 post-infection) (Ref. 14; incorporated by reference in its entirety), the experiments conducted during development of embodiments herein (See, e.g., FIG. 2D) show that a nucleocapsid vaccine facilitates viral control at later times (day 5 post-infection).

### Immunogenicity of a Nucleocapsid Prime-Boost Vaccine Regimen

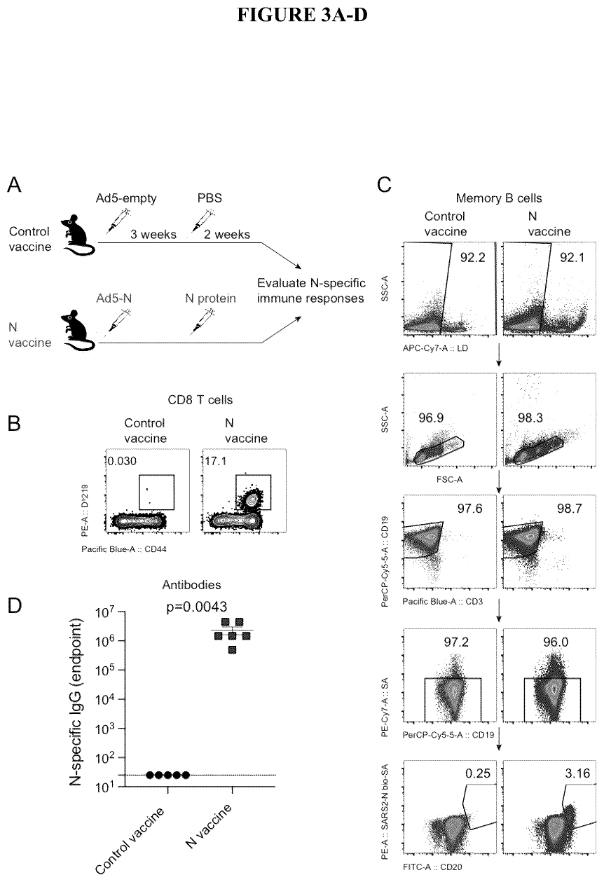

To understand the role of nucleocapsid-specific humoral responses during SARS-COV-2 infection, a prime-boost vaccine regimen was developed that elicited high levels of nucleocapsid-specific antibodies, which were later used in passive immunization experiments. C57BL/6 mice were primed intramuscularly with an adenovirus serotype 5 vector expressing SARS-COV-2 nucleocapsid (Ad5-N) (Refs. 14, 21, 22; incorporated by reference in their entireties) at a dose of 10¹¹ PFU per mouse, followed by booster with 100 μg of nucleocapsid protein three weeks later to generate high titers of nucleocapsid-specific antibody responses. As controls, mice were immunized with an “empty” Ad5 vector (Ad5-Empty) followed by a PBS boost. 2 weeks post-boost, and nucleocapsid-specific immune responses were measured (FIG. 3A). Mice immunized with the nucleocapsid vaccine regimen exhibited robust nucleocapsid-specific CD8 T cell responses (FIG. 3B), memory B cell responses (FIG. 3C), and antibody responses (FIG. 3D).

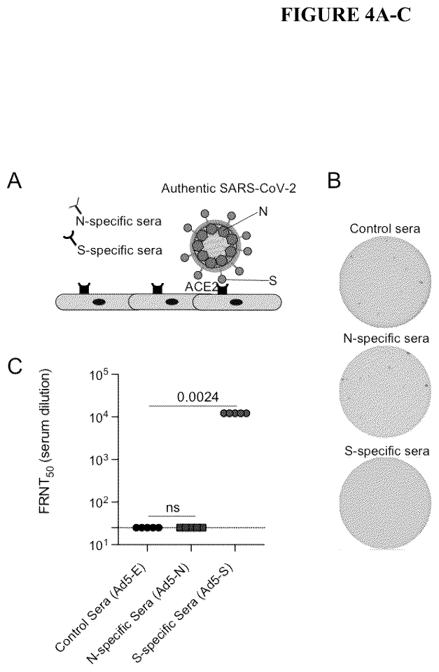

Focus reduction neutralization titer (FRNT) assays were conducted using live SARS-CoV-2 to examine whether nucleocapsid-specific antibodies prevent SARS-COV-2 infection (FIG. 4A). Live SARS-COV-2 was used instead of a pseudovirus, because live virus contains all viral proteins, including nucleocapsid. As positive control, sera from mice that received a spike-based adenovirus vaccine were used (Ad5-S, similar to the CanSino vaccine and the Sputnik vaccine). Sera from mice that received the spike-based vaccine completely prevented SARS-COV-2 infection, even when the sera were diluted 450-fold (FIG. 4B). However, sera from mice that received the nucleocapsid-based vaccine did not exert any antiviral effect in this in vitro infection assay (FIG. 4B-4C). Taken together, only spike-specific (and not nucleocapsid-specific) antibodies can block SARS-COV-2 infection, consistent with the widely established notion that spike-specific antibodies are protective, as they can block the first step in the SARS-COV-2 life cycle: entry into host cells.

### Nucleocapsid-Specific Antibodies Help Clear a SARS-COV-2 Infection In Vivo

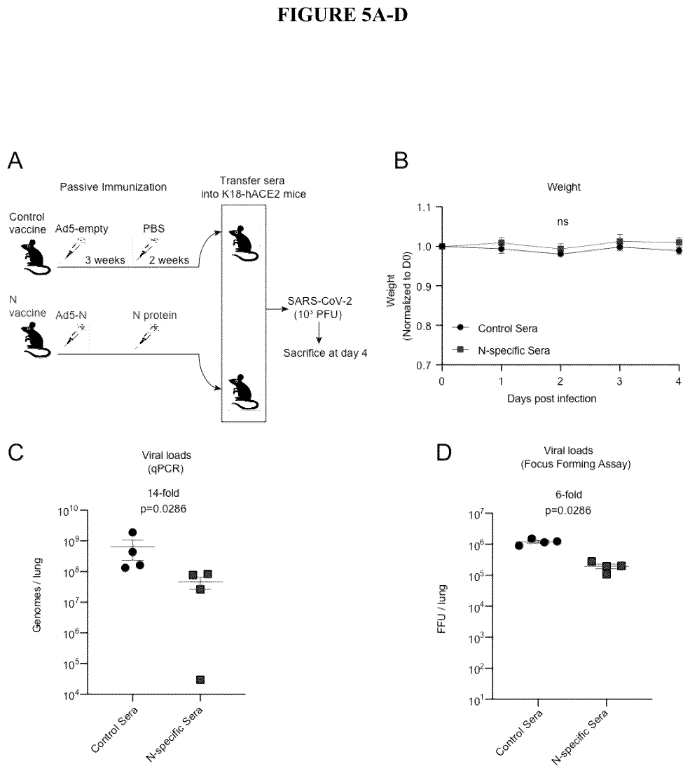

Antibody responses exert antiviral functions by various mechanisms, including viral neutralization and Fc-dependent effector mechanisms. Although nucleocapsid-specific sera did not prevent SARS-COV-2 infection in vitro, it was reasoned that it could confer protection in vivo via effector mechanisms. A passive immunization study was conducted to evaluate whether the specific transfer of nucleocapsid-specific antibodies can confer a clinical benefit (FIG. 5A). 500 μL of nucleocapsid-specific sera was adoptively transferred into naïve K18-hACE2 mice (week 2 post-boost; same mice from FIG. 3A). One day after sera transfer, the K18-hACE2 mice were challenged intranasally with a low dose (10³ PFU) of SARS-COV-2, followed by evaluation of weight loss and viral loads at day 4 post-challenge. Following this low dose viral challenge, weight loss was not observed (FIG. 5B). However, the mice that received nucleocapsid-immune sera showed a significant reduction in viral titers relative to control, by RT-qPCR and focus forming assays (FIG. 5C-5D). These findings demonstrate that even though nucleocapsid-specific humoral responses do not neutralize SARS-COV-2 in vitro, they can exert antiviral effects in vivo.

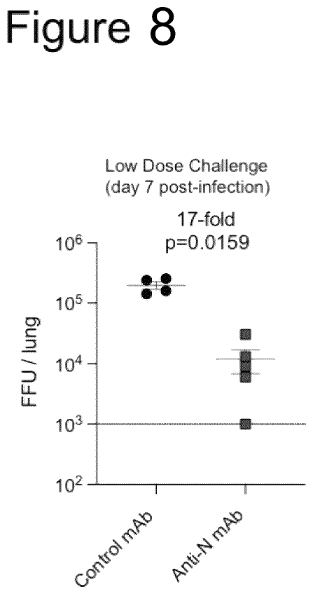

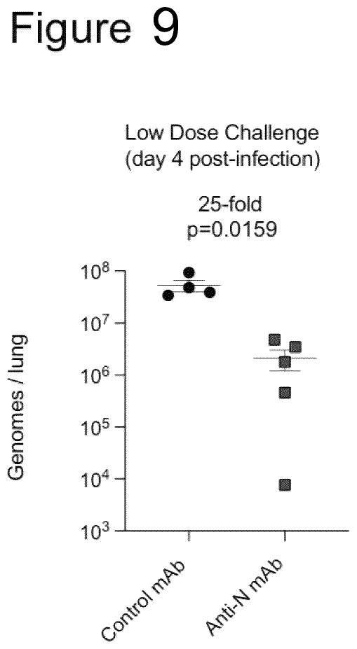

The studies herein indicate that nucleocapsid-specific antibody facilitates clearing of a SARS-COV-2 infection. To ascertain the specific contribution of nucleocapsid-specific antibody, K18-hACE2 mice were treated with a nucleocapsid-specific monoclonal antibody (mAb) or an isotype control antibody, and on the next day, mice received a low dose intranasal SARS-COV-2 challenge (10³) followed by evaluation of viral loads in lungs at day 7 post-infection (FIG. 6A). With this low dose viral challenge, there was no weight loss by day 7 (FIG. 6B). Treatment with nucleocapsid-specific mAb resulted in a significant improvement in viral control by RT-qPCR (163-fold lower viral titers, relative to control) (FIG. 6C). Improved antiviral control in the mice treated with nucleocapsid-specific mAb was also observed by another virological quantification method, focus forming assays (FIG. 8). In separate experiment, lung viral loads were quantified at a more acute time point (day 4) and a similar improvement in viral control was observed in mice that received the nucleocapsid-specific mAb (FIG. 9).

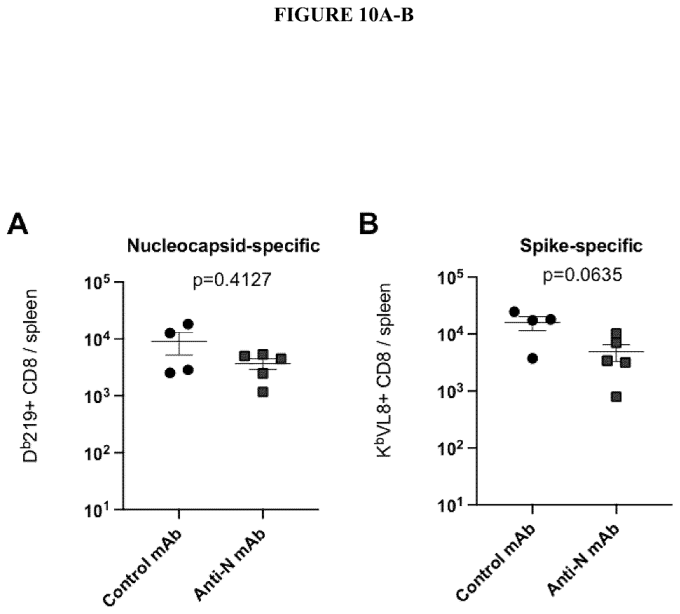

Viral challenges were also performed using a high dose of SARS-COV-2 (5×10⁴ PFU) that normally results in severe weight loss and immunopathology (FIG. 6D). Mice were sacrificed at day 5 post-infection, because this high viral challenge dose causes severe pneumonia and lethality within approximately a week of challenge. Treatment with nucleocapsid-specific mAb mitigated weight loss (FIG. 6E) and resulted in a significant improvement in viral control (FIG. 6F). In addition, mice treated with nucleocapsid-specific mAb exhibited lower levels of inflammatory IL-6 cytokine in circulation (FIG. 6G), and showed reduced lung immunopathology (FIG. 6H-61) relative to control mice. These data demonstrate that nucleocapsid-specific antibodies contribute to the control of a SARS-COV-2 infection and can mitigate COVID-19 disease progression.

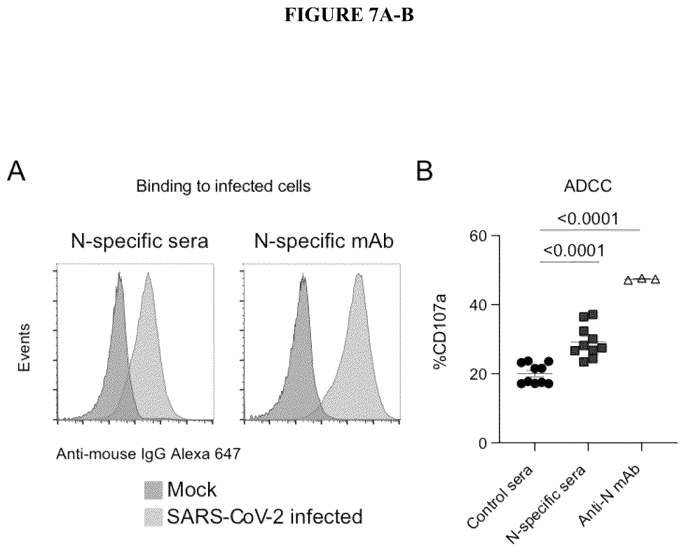

Although nucleocapsid specific antibodies failed to neutralize cell-free SARS-COV-2 in vitro, nucleocapsid is expressed on the surface of SARS-COV-2 infected cells, and therefore has the potential to mediate antibody-dependent cellular cytotoxicity (ADCC) (Refs. 24, 25; incorporated by reference in their entireties)). ADCC assays were conducted using sera from mice immunized with the nucleocapsid vaccine. ADCC activity was examined with the nucleocapsid-specific mAb. It was observed that N-specific antibodies from immune sera or mAb bind to SARS-COV-2 infected cells (FIG. 7A). Moreover, nucleocapsid-specific sera and nucleocapsid-specific mAb triggered ADCC activity by NK cells, evidenced by in vitro CD107a degranulation (FIG. 7B). These findings indicate that nucleocapsid-specific antibodies confer antiviral protection via recognition of infected cells displaying the nucleocapsid antigen on their surface, which triggers Fc-mediated antibody-mediated effector functions.

### REFERENCES

The following references, some of which are cited above by number, are herein incorporated by reference in their entireties.
1. Polack F P, Thomas S J, Kitchin N, Absalon J, Gurtman A, Lockhart S, Perez J L, Perez Marc G, Moreira E D, Zerbini C, et al. Safety and Efficacy of the BNT162b2 mRNA Covid-19 Vaccine. N Engl J Med. 2020; 383(27):2603-15.
2. Baden L R, El Sahly H M, Essink B, Kotloff K, Frey S, Novak R, Diemert D, Spector S A, Rouphael N, Creech C B, et al. Efficacy and Safety of the mRNA-1273 SARS-COV-2 Vaccine. N Engl J Med. 2021; 384(5):403-16.
3. Sadoff J, Gray G, Vandebosch A, Cárdenas V, Shukarev G, Grinsztejn B, Goepfert P A, Truyers C, Fennema H, Spiessens B, et al. Safety and Efficacy of Single-Dose Ad26.COV2.S Vaccine against Covid-19. The New England journal of medicine. 2021.
4. Voysey M, Clemens S A C, Madhi S A, Weckx L Y, Folegatti P M, Aley P K, Angus B, Baillie V L, Barnabas S L, Bhorat Q E, et al. Safety and efficacy of the ChAdOx 1 nCOV-19 vaccine (AZD1222) against SARS-COV-2: an interim analysis of four randomised controlled trials in Brazil, South Africa, and the U K. Lancet. 2021; 397(10269):99-111.
5. Zhu F C, Guan X H, Li Y H, Huang J Y, Jiang T, Hou L H, Li J X, Yang B F, Wang L, Wang W J, et al. Immunogenicity and safety of a recombinant adenovirus type-5-vectored COVID-19 vaccine in healthy adults aged 18 years or older: a randomised, double-blind, placebo-controlled, phase 2 trial. Lancet. 2020; 396(10249):479-88.
6. Logunov D Y, Dolzhikova I V, Shcheblyakov D V, Tukhvatulin A I, Zubkova O V, Dzharullaeva A S, Kovyrshina A V, Lubenets N L, Grousova D M, Erokhova A S, et al. Safety and efficacy of an rAd26 and rAd5 vector-based heterologous prime-boost COVID-19 vaccine: an interim analysis of a randomised controlled phase 3 trial in Russia. Lancet. 2021; 397(10275):671-81.
7. Tande A J, Pollock B D, Shah N D, Farrugia G, Virk A, Swift M, Brecher L, Binnicker M, and Berbari E F. Impact of the COVID-19 Vaccine on Asymptomatic Infection Among Patients Undergoing Pre-Procedural COVID-19 Molecular Screening. Clinical infectious diseases: an official publication of the Infectious Diseases Society of America. 2021.
8. Kechner J, Horton L E, Pfeffer M A, Longhurst C A, Schooley R T, Currier J S, Abeles S R, and Torriani F J. SARS-COV-2 Infection after Vaccination in Health Care Workers in California. The New England journal of medicine. 2021.
9. Levine-Tiefenbrun M, Yelin I, Katz R, Herzel E, Golan Z, Schreiber L, Wolf T, Nadler V, Ben-Tov A, Kuint J, et al. Initial report of decreased SARS-COV-2 viral load after inoculation with the BNT162b2 vaccine. Nature medicine. 2021.
10. Rydyznski Moderbacher C, Ramirez S I, Dan J M, Grifoni A, Hastie K M, Weiskopf D, Belanger S, Abbott R K, Kim C, Choi J, et al. Antigen-Specific Adaptive Immunity to SARS-COV-2 in Acute COVID-19 and Associations with Age and Disease Severity. Cell. 2020; 183(4):996-1012 e19.
11. Lec E, Sandgren K, Ducttc G, Stylianou V V, Khanna R, Eden J S, Blyth E, Gottlieb D, Cunningham A L, and Palmer S. Identification of SARS-COV-2 Nucleocapsid and Spike T-Cell Epitopes for Assessing T-Cell Immunity. J Virol. 2021; 95(6).
12. Grifoni A, Weiskopf D, Ramirez S I, Mateus J, Dan J M, Moderbacher C R, Rawlings S A, Sutherland A, Premkumar L, Jadi R S, et al. Targets of T Cell Responses to SARS-CoV-2 Coronavirus in Humans with COVID-19 Disease and Unexposed Individuals. Cell. 2020; 181(7): 1489-501 e15.
13. Minervina A A, Pogorelyy M V, Kirk A M, Crawford J C, Allen E K, Chou C H, Mettelman R C, Allison K J, Lin C Y, Brice D C, et al. SARS-COV-2 antigen exposure history shapes phenotypes and specificity of memory CD8(+) T cells. Nat Immunol. 2022; 23(5):781-90.
14. Dangi T, Class J, Palacio N, Richner J M, and Penaloza MacMaster P. Combining spike- and nucleocapsid-based vaccines improves distal control of SARS-COV-2. Cell Rep. 2021; 36(10): 109664.
15. Winkler E S, Bailey A L, Kafai N M, Nair S, McCune B T, Yu J, Fox J M, Chen R E, Earnest J T, Keeler S P, et al. SARS-COV-2 infection of human ACE2-transgenic mice causes severe lung inflammation and impaired function. Nature immunology. 2020; 21(11): 1327-35.
16. Oladunni F S, Park J G, Pino P A, Gonzalez O, Akhter A, Allue-Guardia A, Olmo-Fontanez A, Gautam S, Garcia-Vilanova A, Ye C, et al. Lethality of SARS-COV-2 infection in K18 human angiotensin-converting enzyme 2 transgenic mice. Nat Commun. 2020; 11(1):6122.
17. Rosenfeld R, Noy-Porat T, Mechaly A, Makdasi E, Levy Y, Alcalay R, Falach R, Aftalion M, Epstein E, Gur D, et al. Post-exposure protection of SARS-COV-2 lethal infected K18-hACE2 transgenic mice by neutralizing human monoclonal antibody. Nat Commun. 2021; 12(1):944.
18. Zheng J, Wong L R, Li K, Verma A K, Ortiz M E, Wohlford-Lenane C, Leidinger M R, Knudson C M, Meyerholz D K, McCray P B, Jr., et al. COVID-19 treatments and pathogenesis including anosmia in K18-hACE2 mice. Nature. 2021; 589(7843):603-7.
19. Bao L, Deng W, Huang B, Gao H, Liu J, Ren L, Wei Q, Yu P, Xu Y, Qi F, et al. The pathogenicity of SARS-COV-2 in hACE2 transgenic mice. Nature. 2020; 583(7818):830-3.
20. Matchett W E, Joag V, Stolley J M, Shepherd F K, Quarnstrom C F, Mickelson C K, Wijeyesinghe S, Soerens A G, Becker S, Thiede J M, et al. Nucleocapsid Vaccine Elicits Spike-Independent SARS-COV-2 Protective Immunity. J Immunol. 2021.
21. Joag V, Wijeyesinghe S, Stolley J M, Quarnstrom C F, Dileepan T, Soerens A G, Sangala J A, O'Flanagan S D, Gavil N V, Hong S W, et al. Cutting Edge: Mouse SARS-COV-2 Epitope Reveals Infection and Vaccine-Elicited CD8 T Cell Responses. J Immunol. 2021; 206(5):931-5.
22. Dangi T, Palacio N, Sanchez S, Park M, Class J, Visvabharathy L, Ciucci T, Koralnik I J, Richner J M, and Penaloza-MacMaster P. Cross-protective immunity following coronavirus vaccination and coronavirus infection. J Clin Invest. 2021; 131(24).
23. Sanchez S, Palacio N, Dangi T, Ciucci T, and Penaloza-MacMaster P. Fractionating a COVID-19 Ad5-vectored vaccine improves virus-specific immunity. Sci Immunol. 2021; 6(66):cabi8635.
24. Lopez-Munoz A D, Kosik I, Holly J, and Yewdell J W. Cell surface SARS-COV-2 nucleocapsid protein modulates innate and adaptive immunity. Sci Adv. 2022; 8(31):cabp9770.
25. Fielding C A, Sabberwal P, Williamson J C, Greenwood E J D, Crozier T W M, Zelek W, Scow J, Graham C, Huettner I, Edgeworth J D, et al. SARS-COV-2 host-shutoff impacts innate N K cell functions, but antibody-dependent N K activity is strongly activated through non-spike antibodies. Elife. 2022; 11(
26. Corey L, Gilbert P B, Juraska M, Montefiori D C, Morris L, Karuna S T, Edupuganti S, Mgodi N M, deCamp A C, Rudnicki E, et al. Two Randomized Trials of Neutralizing Antibodies to Prevent HIV-1 Acquisition. N Engl J Med. 2021; 384(11): 1003-14.
27. Sneller M C, Blazkova J, Justement J S, Shi V, Kennedy B D, Gittens K, Tolstenko J, McCormack G, Whitehead E J, Schneck R F, et al. Combination anti-HIV antibodies provide sustained virological suppression. Nature. 2022; 606(7913):375-81.
28. Zeng C, Evans J P, King T, Zheng Y-M, Oltz E M, Whelan S P J, Saif L J, Peeples M E, and Liu S-L. SARS-COV-2 spreads through cell-to-cell transmission. Proceedings of the National Academy of Sciences. 2022; 119(1):e2111400119.
29. Casadevall A, Dragotakes Q, Johnson P W, Senefeld J W, Klassen S A, Wright R S, Joyner M J, Paneth N, and Carter R E. Convalescent plasma use in the USA was inversely correlated with COVID-19 mortality. Elife. 2021; 10(
30. Jabal K A, Wiegler K B, and Edelstein M. Convalescent plasma from people vaccinated after COVID-19 infection. Lancet Microbe. 2021; 2(5):e171-e2.
31. Dangi T, Chung Y R, Palacio N, and Penaloza-MacMaster P. Interrogating Adaptive Immunity Using LCMV. Curr Protoc Immunol. 2020; 130(1):e99.
32. Palacio N, Dangi T, Chung Y R, Wang Y, Loredo-Varela J L, Zhang Z, and Penaloza-MacMaster P. Early type I IFN blockade improves the efficacy of viral vaccines. J Exp Med. 2020; 217(12).
33 Rihn S J, Merits A, Bakshi S, Turnbull M L, Wickenhagen A, Alexander A J T, Baillie C, Brennan B, Brown F, Brunker K, et al. A plasmid DNA-launched SARS-COV-2 reverse genetics system and coronavirus toolkit for COVID-19 research. PLOS Biol. 2021; 19(2):e3001091.
34 Aguilar O A, Fong L K, Ishiyama K, DeGrado W F, and Lanier L L. The CD3zeta adaptor structure determines functional differences between human and mouse CD16 Fc receptor signaling. J Exp Med. 2022; 219(5).

SEQUENCES

SEQ ID NO: 1 (SARS-CoV-2 nucleocapsid protein)
msdngpqnqrnapritfggpsdstgsnqngersgarskqrrpqglpnnt
aswftaltqhgkedlkfprgqgvpintnsspddqigyyrratrrirggd
gkmkdlsprwyfyylgtgpeaglpygankdgiiwvategalntpkdhig
trnpannaaivlqlpqgttlpkgfyaegsrggsqassrsssrsrnssrn
stpgssrgtsparmagnggdaalalllldrlnqleskmsgkgqqqqgqt
vtkksaaeaskkprqkrtatkaynvtqafgrrgpeqtqgnfgdqelirq
gtdykhwpqiaqfapsasaffgmsrigmevtpsgtwltytgaiklddkd
pnfkdqvillnkhidayktfpptepkkdkkkkadetqalpqrqkkqqtv
tllpaadlddfskqlqqsmssadstqa
SEQ ID NO: 2. GAC CCC AAA ATC AGC GAA AT
SEQ ID NO: 3. TCT GGT TAC TGC CAG TTG AAT CTG
SEQ ID NO: 4. ACC CCG CAT TAC GTT TGG TGG ACC

1. (canceled)

2. The method of claim 9, wherein the pharmaceutical composition is formulated for oral or parenteral administration.

3. The method of claim 9, wherein the antibody or antigen-binding fragment is a monoclonal antibody.

4. The method of claim 9, wherein the antibody or antigen-binding fragment is a human antibody.

5. The method of claim 9, wherein the antibody or antigen-binding fragment is a IgG1, IgG2, IgG3, or IgG4 class.

6. The method of claim 9, wherein the antibody or antigen-binding fragment is a Fab fragment, Fv fragment, or single-chain Fv antibody.

7-8. (canceled)

9. A method of treating or increasing the rate of clearance of a SARS-COV-2 infection in a subject comprising administering a pharmaceutical composition comprising administering a pharmaceutical composition comprising an antibody or antigen-binding fragment capable of binding to a SARS-CoV-2 nucleocapsid protein to the subject.

10. The method of claim 9, wherein the subject is infected with SARS-COV-2.

11. The method of claim 10, wherein the subject exhibits symptoms of SARS-CoV-2 infection.

12. The method of claim 9, wherein the subject is at risk of infection with SARS-CoV-2.

13. The method of claim 9, wherein the pharmaceutical composition is co-administered with one or more additional therapeutic or prophylactic agents.

14. A method of improving viral clearance of SARS-COV-2 from a subject comprising vaccinating the subject against a SARS-COV-2 nucleocapsid protein or an antigen or epitope thereof.

15. The method of claim 14, wherein the subject is not infected with SARS-COV-2.

16. The method of claim 15, wherein the vaccination improves the rate of viral clearance for future SARS-COV-2 infections for the subject.

17-21. (canceled)
